(19) United States
(12) Patent Application Publication
(10) Pub. No.: US 2021/0128651 A1
(43) Pub. Date: May 6, 2021

(54) SURFACE FUNCTIONALIZATION OF PROBIOTICS AND APPLICATIONS THEREOF

(71) Applicant: The University of North Carolina at Chapel Hill, Chapel Hill, NC (US)

(72) Inventors: Aaron ANSELMO, Chapel Hill, NC (US); Ava VARGASON, Chapel Hill, NC (US)

(21) Appl. No.: 17/086,967

(22) Filed: Nov. 2, 2020

Related U.S. Application Data
(60) Provisional application No. 62/929,234, filed on Nov. 1, 2019.

(51) Int. Cl. A61K 35/747; A61K 35/741; A61K 35/742; A61K 31/4188

(52) U.S. Cl. CPC A61K 35/747; A61K 31/4188; A61K 35/742; A61K 35/741

(57) ABSTRACT

In one aspect, probiotic compositions are described herein comprising surface modified microbes operable to adhere or bind to surfaces of the gastrointestinal tract. In some embodiments, for example, a composition for enhancing gastrointestinal health comprises microbes modified with one or more surface moieties, the surface moieties comprising functionality for binding the modified microbes to surfaces of the gastrointestinal tract.

### RELATED APPLICATION DATA

The present application claims priority pursuant to 35 U.S.C. § 119(e) to U.S. Provisional Patent Application Ser. No. 62/929,234 filed Nov. 1, 2019 which is incorporated herein by reference in its entirety.

### FIELD

The present invention relates to probiotic compositions and, in particular, to probiotic compositions comprising surface functionalized microbes for enhancing microbial adhesion or binding to gastrointestinal surfaces.

### BACKGROUND

The human body exists in a symbiotic relationship with a diverse community of bacteria, viruses and fungi that are collectively called the microbiome. The microbiome plays essential roles in human health, including critical metabolic, immune and anti-virulence functions. In particular, commensal bacteria are involved in the exclusion of pathogens from the human gastrointestinal tract by secreting antimicrobial compounds, competing with pathogens for nutrients, activating the immune system, and physically preventing the attachment of pathogens to mammalian tissues and cells. Collectively, these actions grant the host colonization resistance against potentially deadly pathogens. For this reason, research into therapeutic bacteria that enhance colonization resistance, such as biotherapeutics or probiotics, is increasing.

### SUMMARY

In one aspect, probiotic compositions are described herein comprising surface modified microbes operable to adhere or bind to surfaces of the gastrointestinal tract. In some embodiments, for example, a composition for enhancing gastrointestinal health comprises microbes modified with one or more surface moieties, the surface moieties comprising functionality for binding the modified microbes to surfaces of the gastrointestinal tract. The microbes can be synthetically modified by covalently attaching one or more surface moieties to the microbes. Alternatively, the one or more surface moieties can be non-covalently attached to the microbes. Non-covalent attachment to the microbes can be achieved through a variety of interactions including ionic interactions, van der Waals interactions, hydrogen bonding, hydrophobic interactions and/or hydrophilic interactions.

Surfaces of the gastrointestinal tract to which the modified microbes can bind via the surface moieties include, but are not limited to, epithelial cells, mucus, unmodified microbes in the gastrointestinal tract, and combinations thereof. In some embodiments, surface moieties of the modified microbes covalently bind with surfaces of the gastrointestinal tract. In other embodiments, the microbial surface moieties non-covalently bind with surfaces of the gastrointestinal tract. Non-covalent binding of the modified microbes to surfaces of the gastrointestinal tract can occur via several interactions including, but not limited to, ionic interactions, van der Waals interactions, hydrogen bonding, hydrophobic interactions and/or hydrophilic interactions. In some embodiments, surface moieties of the modified microbes exhibit functionalities for specific or targeted binding interactions with gastrointestinal surfaces. The surface moieties, in some embodiments, can exhibit specific or targeted binding to receptors and/or other chemical architectures of cells and/or other chemical species forming surfaces of the gastrointestinal tract. Surface moieties of the modified microbes exhibiting targeting or specific binding functionalities can include polymeric species, antibodies, peptides, aptamers, fats, metabolites, peptidomimetics, and combinations thereof.

In other embodiments, surface moieties of the modified microbes exhibit non-specific or non-targeted binding interactions with surfaces of the gastrointestinal tract. Non-specific or non-targeted binding interactions can be covalent or non-covalent interactions.

Surface moieties and/or modifications to microbes described herein can include the attachment or binding of polymers (e.g. mucoadhesive chitosan), antibodies (e.g. ICAM antibodies to target inflammation), peptides (e.g. peptides that target specific pathogenic microbes), aptamers (e.g. aptamers to target epithelial cells), food-derived molecules (e.g. tomato lectins for epithelial binding or fructose for nutritional supplement of the bacteria), small molecule ligands (e.g. peptidomimetics or metabolites for targeting of epithelial cells) and either intact cell membranes from epithelial cells and bacteria or components of cell membranes (e.g. isolated bacterial adhesins for adhesion to GI lining) to mimic natural functions of mammalian or bacterial membranes.

As described herein, surface moieties and/or modification to microbes can rely on intermolecular forces between the entities on the microbe surface and different components of the delivery microenvironment such as the mucus, epithelial cells, other microbes naturally present in the microbiome, and lumen contents such as food. Examples of intermolecular forces that can mediate these interactions include electrostatic/ionic interactions (e.g. positively-charged chitosan binding to negatively charged bacteria or mammalian-cell membranes), covalent bonding (e.g. poly(acrylic acid) binding to mucus glycoproteins), van der Waals forces (e.g. non-specific protein-protein interactions; a specific example are the highly-charged discrete sections of targeted antibodies interacting with highly-charged discrete sections of non-target proteins), hydrogen bonding (e.g. pectin-Mucin), hydrophobic attraction (e.g. hydrophobic polymers binding to mucins), steric Repulsion (e.g. dense PEG coatings to displace water on the molecular scale to better facilitate diffusion through mucus), and receptor-ligand interactions (e.g. antibody-antigen receptor).

Surface moieties can be attached to bacteria/microbes either through specific or non-specific interactions. Specific interactions include bioconjugation reactions such as amine-carboxylate couplings (including reaction of isothiocyanates, tetraphluorphenyl esters, succinimidyl esters, sulfodichlorophenol esters with amines), thiol-Maleimide reactions (using thiol groups on the surface of bacteria), and carbodiimide reactions (using thioureas or isocyanate intermediate groups). Bacterial and/or microbial surfaces can also be functionalized using hydrazide-aldehyde crosslinking reactions following aldehyde-activation of the bacterial surface with periodic acid. Similarly, carbonyl groups on the surface of bacteria/microbes can be activated to ketones or aldehydes and crosslinked with alkoxyamine compounds. Biocompatible click-chemistry reactions such as copper-catalyzed azide-alkyne cycloaddition or strain-promoted azide-aklyne cycloaddition can also be used. Another surface modification approach are condensation reactions that include hydrazone formation using aniline. Finally, non-specific interactions that rely on the intermolecular forces described above can also be used to non-specifically adsorb or attach entities to the surface of bacteria; for example hydrophobic-hydrophobic attraction to bacterial cell walls (polymers, lipids) or electrostatic/ionic attractions of positively charged entities to negatively charged cell walls.

Synthetically modified microbes described herein can comprise any type or species of microbe not inconsistent with the objectives of enhancing or improving gastrointestinal health. In some embodiments, for example, the modified microbes comprise bacteria, fungi, viruses, protozoa, algae, archaea or mixtures thereof.

In another aspect, methods of treating gastrointestinal surfaces are described herein. Such methods can be employed to treat one or more gastrointestinal conditions and/or promote or enhance gastrointestinal health of an individual. In some embodiments, a method of treating gastrointestinal surfaces comprises modifying microbes with one or more surface moieties, and delivering the modified microbes to the gastrointestinal tract of an individual. The modified microbes bind to the gastrointestinal surfaces via the one or more surface moieties. The modified microbes can comprise any surface moieties and/or binding characteristics described above, including covalent, non-covalent, specific or non-specific. Surface moieties, for example, can comprise functionality to enhance binding of the modified microbes to gastrointestinal surfaces relative to one or more unmodified microbial species. Accordingly, modified microbes having structure and functionality described herein can be used to block pathogenic attachment to surfaces of the gastrointestinal tract. The modified microbes, for example, can block specific receptor sites and/or generally compete with pathogenic species at various non-specific surface sites in the gastrointestinal tract.

These and other embodiments are further described in the following detailed description.

### BRIEF DESCRIPTION OF THE DRAWINGS

FIG. 1A is a schematic of N-hydroxysuccinimide ester chemistry for bioconjugation of biotin to primary amines on the bacteria surface, according to some embodiments.

FIG. 1B illustrates viability of various bacterial species prior to and following biotinylation reaction (error represents standard deviation, n=3, significance assessed using multiple unpaired Student's t-tests).

FIG. 1C provides epi-fluorescence images of unmodified (top) and biotinylated (bottom) Lactobacillus casei (LC) following incubation with Alexa Fluor® Streptavidin Conjugate.

FIG. 1D are scanning electron microscopy images of unmodified (top) and biotinylated (bottom) LC.

FIG. 1E illustrates results of growth studies of biotinylated and unmodified Escherichia coli (EC), LC and Bacillus coagulans (BC) (error represents standard deviation, n=3). Epi-fluorescence scale bar=50 μm. SEM scale bars=1 μm.

FIG. 2A are representative images of unmodified (top) and biotinylated (bottom) bacteria at increasing optical densities (OD600), according to some embodiments.

FIG. 2B provides quantification of the concentration-dependent attachment of unmodified and biotinylated bacteria to a streptavidin-coated well-plate (error represents standard deviation, n=3, significance assessed using multiple unpaired Student's t-tests, ** p<0.01).

FIG. 2C is a schematic of streptavidin conjugation to the constant region of IgG antibodies, thereby enabling antibody attachment to the surface of biotinylated bacteria (abbreviated as LTB=live therapeutic bacteria).

FIG. 2D quantifies attachment of unmodified and biotinylated bacteria to a monolayer of Caco-2 cells after no incubation or incubation with an anti-ICAM antibody (aICAM) or anti-ICAM-streptavidin conjugate (aICAM-streptavidin) (error represents standard deviation, n=3, significance assessed using two-way ANOVA with Sidak's multiple comparisons, **p<0.01).

FIG. 2E are representative images of bacteria attached to Caco-2 monolayers. Scale bars=(A) 130 μm and (E) 65 μm.

FIG. 3A illustrates quantification of Escherichia coli (EC) attachment to Caco-2 cells under three conditions: no probiotic pre-treatment (left), pre-treatment with unmodified Lactobacillus casei (LC) (middle) and pre-treatment with ICAM-targeted LC (right).

FIG. 3B quantifies CFU of attached unmodified and ICAM-targeted (aICAM) LC to Caco-2 monolayers via plating following 1 hour of incubation (error represents standard deviation, n=4, significance assessed using unpaired Student's t-test, *** p<0.001).

FIG. 3C quantifies EC attachment following pre-incubation with unmodified or ICAM-targeted (aICAM) LC for 1 hour, followed by a 1 hour challenge with EC. Results are normalized to the amount of EC attached without pre-incubation (error represents standard deviation, n>15 with at least 5 images per well and 3 wells per conditions, significance assessed using two-way ANOVA with Sidak's multiple comparisons, ***p<0.001).

FIG. 3D are representative images following challenge with GFP-expressing EC. Scale bar=65 μm.

FIG. 4A illustrates study parameters of eight-week old female BALB/c mice that were treated with streptomycin for 24 hours, followed by an 18-hour wash-out period. Mice were treated with unmodified or aMUC2 synthetic adhesin-(SA-EcN) Escherichia coli Nissle 1918 (EcN) via oral gavage and fecal pellets were collected at indicated timepoints.

FIG. 4B illustrates determination of viable colony forming units (CFU) of EcN in feces by homogenizing and plating fecal pellets at indicated timepoints for unmodified (purple) and synthetic adhesin (green) EcN (bars represent median, n=5, significance assessed using two-way ANOVA with Sidak's multiple comparisons, *p<0.05).

FIG. 4C provides kinetics of colonization, defined as detectable EcN in feces (n=5, significance assessed using Log-rank Mantel-Cox test, *p<0.05).

FIG. 4D provides time to colonization for each mouse, defined as detectable EcN in feces (error represents standard deviation, n=5, significance assessed using unpaired Student's t-test).

FIGS. 4E, 4F, and 4G detail pharmacokinetics of EcN colonization in the murine GI tract, including (FIG. 4E) maximum detected CFU in each animal, (FIG. 4F) the time CFUmax occurred in each animal and (FIG. 4G) the area under the log(CFU g⁻¹)-time curve for each animal (error represents standard deviation, n=5, significance assessed using unpaired Student's t-tests, *p<0.05, n.s.=not significant)

FIG. 5A illustrates study parameters of eight-week old female BALB/c mice dosed with unmodified or aMUC2 synthetic adhesin-modified (SA-EcN) EcN via oral gavage and sacrificed 1-, 4-, 24- or 72-hours later. Intestines were harvested and EcN abundance was evaluated by plating.

FIG. 5B details abundance of EcN in the small intestine (SI), cecum, and colon of mice 1- (left) and 4-hours (right) post-gavage (error represents standard deviation, n=5, significance assessed using multiple unpaired Student's t-tests, *p<0.05).

FIG. 5C details abundance of EcN in SI, cecum, and colon of mice 24- (left) and 72-hours (right) post-gavage (error represents standard deviation, n=5, significance assessed using multiple unpaired Student's t-tests, no significant differences between groups).

FIG. 5D details concentration of EcN in feces and entire intestinal tract from mice with no viable counts in their feces (noncolonized, left) and viable counts in their feces (colonized, right) (bars represent median). Abundance is dose-normalized to account for variations [Dose-Normalized log(CFU g⁻¹)=log(CFU g⁻¹) Detected in Organ/log(Dose Administered)] in (B-C).

FIG. 6A details Binding of a fluorescent streptavidin probe by unmodified (green) or biotinylated (pink) Bacillus coagulans (BC), Lactobacillus casei (LC), Escherichia coli Nissle (EcN), and E. coli DH5a (DH5a), quantified on a microplate reader.

FIG. 6B are representative images of fluorescent streptavidin probe bound on the surface of biotinylated (top) or control (bottom) live biotherapeutic products (LBPs). (n=3, error shown as standard deviation, significance assessed using multiple unpaired Student's t-tests, α=0.05). Scale bar=30 μm.

FIG. 7A illustrates the growth and corresponding biotin coverage of modified E. coli DH5α determined at varying timepoints (top) and attachment to a streptavidin-coated well-plate was assessed at each timepoint relative to an unmodified control (bottom).

FIG. 7B details biotin concentration on the LBP surface (circles) during growth (squares), measured using a fluorescent streptavidin probe and normalized per colony forming unit (CFU) of bacteria.

FIG. 7C quantifies attachment efficiency of biotinylated or unmodified LBPs after indicated timepoints of growth (Attachment Efficiency=Fluorescent Signal_{Post-Wash}/Fluorescent Signal_{Pre-Wash}*100).

FIG. 7D are representative images of attached biotinylated (top) and unmodified (bottom) LBPs on the well plate floor. (n=3, error shown as standard deviation, significance assessed using two-way ANOVA with Sidak's post hoc test for multiple comparisons, α=0.05, ***p<0.001, *p<0.05, ns=not significant). Scale bar=65 μm.

FIG. 8A illustrates biotinylated or unmodified E. coli DH5α incubated on a streptavidin-coated well-plate for 20-minutes at varying concentrations. Attachment was assessed using fluorescence intensity and images of the well-plate floor after washing.

FIG. 8B details attachment of biotinylated and unmodified LBPs at varying concentrations. Images were quantified using ImageJ. (N=18, with 3 images per well and 6 wells per condition).

FIG. 8C are representative images of biotinylated (top) and unmodified (bottom) LBPs. (bars represent median, significance assessed using two-way ANOVA with Sidak's post hoc test for multiple comparisons, α=0.05, ***p<0.001, ns=not significant). Scale bar=65 μm.

FIG. 9A quantifies attachment of unmodified and biotinylated E. coli DH5α following incubation on a streptavidin-coated well-plate for indicated timepoints in PBS at 4° C.

FIG. 9B are representative images of attached biotinylated (top) or unmodified (bottom) LBPs at varying timepoints. (bars represent median, N=9, with 3 images per well and 3 wells per condition, significance assessed using two-way ANOVA with Sidak's post hoc test for multiple comparisons, α=0.05, ***p<0.001, ns=not significant). Scale bar=65 μm.

FIG. 10A details growth of Bacillus coagulans (BC), Lactobacillus casei (LC), E. coli Nissle 1917 (EcN), and E. coli DH5α (DH5a) before and after biotinylation.

FIG. 10B provides LBP viability assessed as colony forming units (CFU) of BC, LC, EcN, and DH5a immediately prior to and after biotinylation.

FIG. 10C provides viability of unmodified or biotinylated EcN following storage at −80° C. in 25% glycerol solution.

FIG. 10D are representative images of biotinylated (top) and unmodified (bottom) EcN binding a fluorescent streptavidin probe after one week of storage. (n=3, bars or shading represent standard deviation, significance assessed using multiple unpaired Student's t-tests with Holm-Sidak's post hoc test for multiple comparisons in B or two-way ANOVA with Sidak's post hoc test for multiple comparisons in C, ns=not significant). Scale bar=15 μm.

FIG. 11A provides viability of Caco-2 cells following incubation with unmodified (green) or biotinylated (pink) Escherichia coli Nissle 1917 or Lactobacillus casei for one (white) or two (grey) hours, measured using an MTT assay. (n=3, bars represent standard deviation).

FIG. 11B details L-Lactate production in picomolar (pM) units from L. casei, normalized per colony forming unit (CFU) of bacteria in MRS media. (n=3, bars represent standard deviation).

FIG. 11C provides eight-week old female BALB/c mice were treated with 10⁸ CFU of unmodified or biotinylated EcN and fecal pellets were collected at indicated timepoints. Abundance of EcN was assessed by homogenizing fecal samples, plating on selective agar plates, and enumerating viable CFU. (n=5, bars represent median, limit of detection (LOD)=3, values below LOD are shown as LOD/2).

FIG. 11D quantifies rate of EcN colonization, defined as the day at which detectable EcN was present in the feces of individual mice (n=5). (significance assessed using a two-way ANOVA with Sidak's post hoc test for multiple comparisons with α=0.05 in A-C or Log-rank Mantel-Cox test in D, **p<0.01, ns=not significant).

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

Compositions described herein comprise surface modified microbes operable to adhere or bind to surfaces of the gastrointestinal tract. Surface modification of the microbes, in some embodiments, can improve and/or enhance microbe colonization, maximum concentration, and/or time to maximum concentration in the gastrointestinal tract.

### EXAMPLE 1

### Surface Modified Live Therapeutic Bacteria

As described herein, microbe surfaces can be modified with various moieties. In the present example, biocompatible ester-amine chemistry is employed to conjugate synthetic adhesins to surfaces of microbes of live therapeutic bacteria (LTB). With such bacterial surface modifications improved attachment of the bacteria to abiotic surfaces, monolayers of mammalian cells, and the mouse GI tract is demonstrated. These surface modifications: (i) are non-toxic to live bacteria, (ii) can be applied to any synthetic adhesin target or LTB species or consortia and (iii) translate to enhanced in vitro and in vivo LTB performance due to improved colonization kinetics in the GI tract.

Specifically, it is shown that prophylactic treatment of mammalian cells with surface modified LTBs significantly improves their colonization resistance, resulting in decreased pathogen attachment. Additionally, using fecal samples as a proxy for intestinal LTB concentration, it was found that synthetic adhesins improve the in vivo pharmacokinetics of LTBs, including their rate of colonization, maximum concentration, and the total exposure over time. It was further confirmed that fecal samples are an accurate representation of intestinal LTB concentration and tracked viable LTB load in the intestinal tract and feces of mice to determine the effect of synthetic adhesins on both short-term intestinal LTB transit and longer-term niche formation. Altogether, the data presented herein demonstrates that the pharmacokinetic improvement provided by synthetic adhesins is a result of an initial increased abundance in the small intestine and cecum, leading to improved niche formation along the intestinal tract in the 3-days post-administration. This technology represents a rapid, tunable approach that can address colonization challenges by controlling specific interactions between the LTB and its adhesion target.

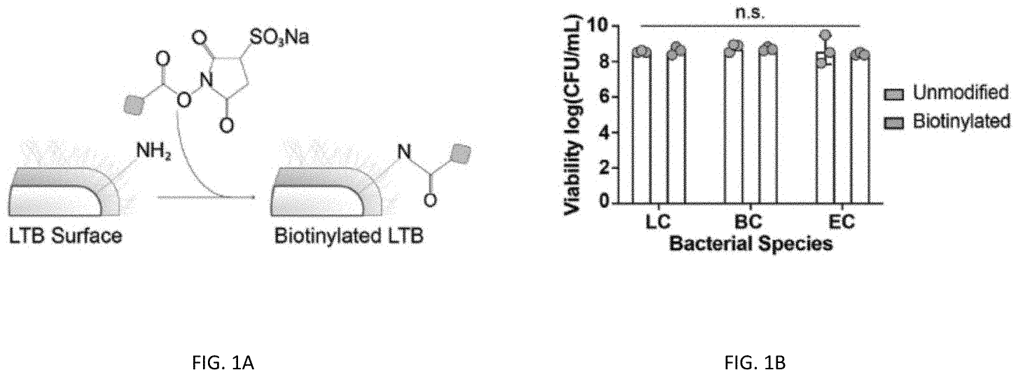

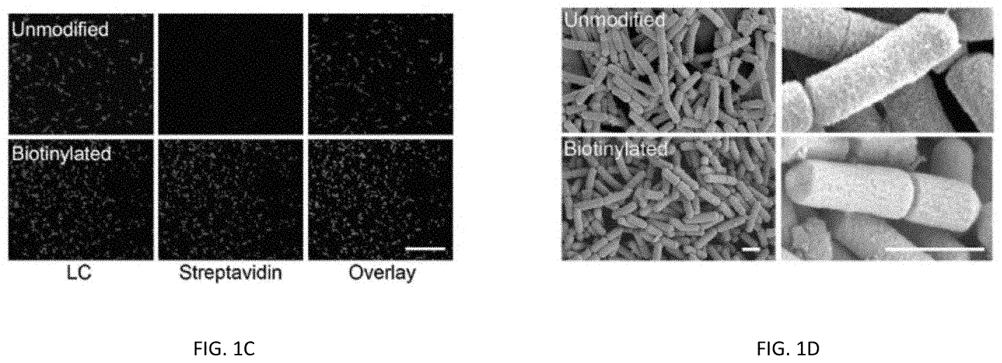

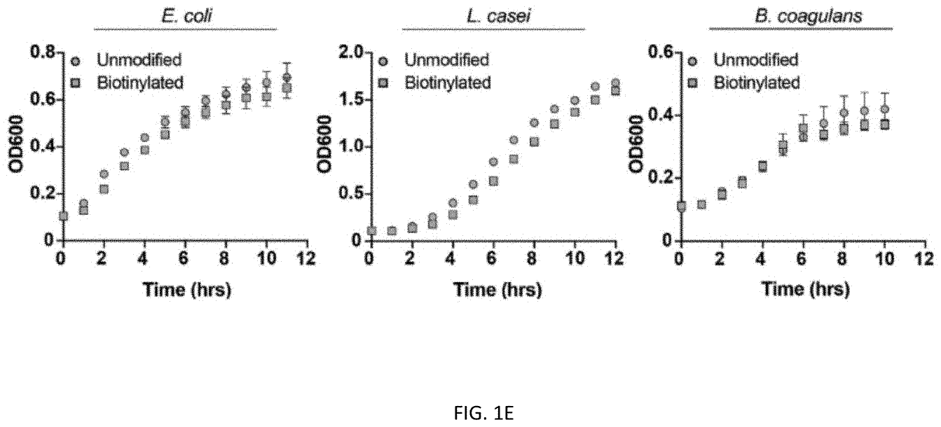

To demonstrate the feasibility and modularity of chemically conjugating synthetic adhesins to the surface of live bacteria, biotin was conjugated to the surface of three bacterial species: Lactobacillus casei (LC), Escherichia coli (EC), and Bacillus coagulans (BC). Biotin was conjugated to the surface of bacteria using N-hydroxysuccinimide ester (NHS) chemistry, which reacts with ubiquitous primary amines on bacterial surfaces (FIG. 1A) to form amide bonds. The viability of each species was unaltered following biotinylation, demonstrating that NETS-ester chemistry is non-toxic to bacteria (FIG. 1B). Biotinylation was confirmed and quantified using a fluorescent streptavidin probe that selectively bound to the surface of biotinylated bacteria (FIG. 1C). Fluorescence from biotin-bound streptavidin probes was quantified using a microplate reader, revealing that the three bacterial species demonstrate differences in the extent of surface biotinylation. These differences may be attributed to varying primary amine densities, surface charges, and total surface area between the three bacterial species. It was further confirmed the modularity of this approach by applying surface modifications to the commercially available probiotic consortia VISIBIOME®. Following biotinylation, streptavidin was able to bind bacterial species in the VISIBIOME® consortium with high specificity compared to an unmodified control. Scanning electron microscopy (SEM) revealed no signs of morphological differences between the unmodified and biotinylated bacteria (FIG. 1D), a standard indicator of bacterial damage to the cell wall. Finally, it was demonstrated that the growth behavior for all strains was not affected by biotinylation (FIG. 1E).

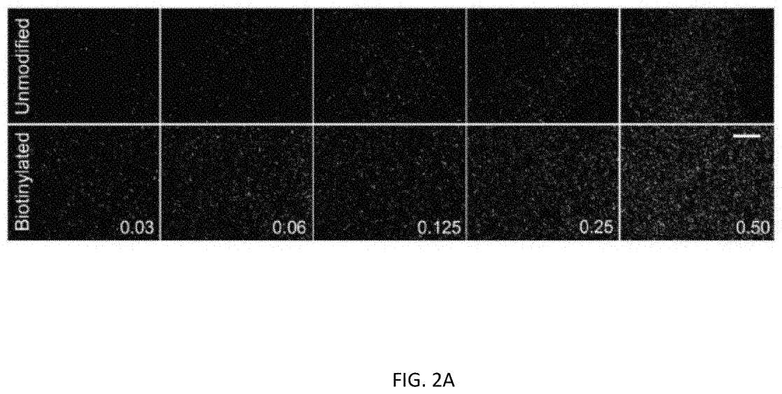

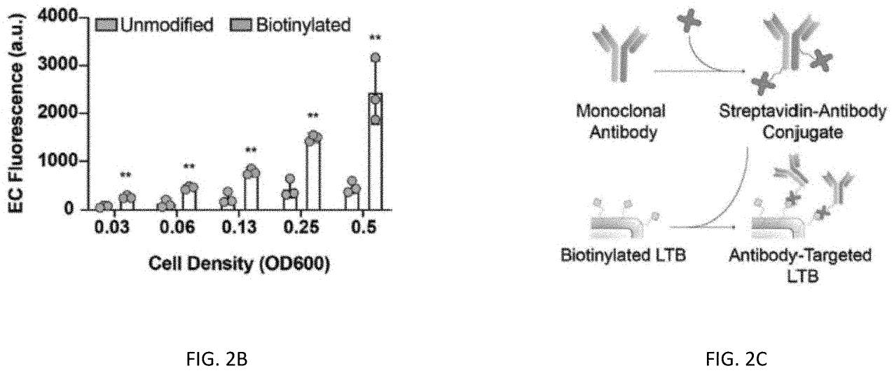

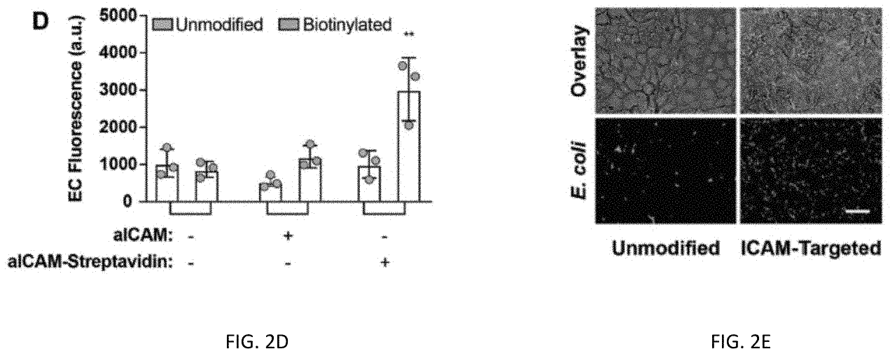

To determine whether biotinylation of bacteria significantly alters their attachment to surfaces, bacterial attachment to an abiotic streptavidin-coated well-plate and to monolayers of mammalian cells were quantified. For these studies, an engineered strain of EC DH5α expressing GFP was used to quantify attachment of bacteria. Both biotinylated and unmodified bacteria were incubated on a streptavidin-coated plate for 1 hour at varying concentrations. Following washes, biotinylated bacteria attached at significantly higher quantities than unmodified bacteria for all concentrations tested (FIGS. 2A and 2B). The attachment of biotinylated bacteria showed a strong, dose-dependent and linear relationship (FIG. 2B).

In the GI tract, probiotic bacteria must adhere to human tissue, mucus or cells to prevent mechanical clearance due to peristalsis and mucus turnover. To enhance the adherence of biotherapeutics to mammalian cells, monoclonal antibodies were attached to the surface of biotinylated bacteria by conjugating streptavidin groups to the constant region of the antibody (FIG. 2C). Antibody conjugation was confirmed using a native protein gel and attachment of the conjugate to the bacterial surface using fluorescence and zeta potential, which has previously been used to assess bacterial surface charge and confirm surface modifications. To target the carcinoma cell-line Caco-2, which is frequently used as a model of the intestinal barrier, streptavidin was conjugated to a monoclonal antibody against Intracellular Adhesion Molecule (aICAM-1), to specifically bind to surface-expressed ICAM-1 receptors on Caco-2 cells. ICAM-targeted and unmodified bacteria were incubated with Caco-2 cells for 1 hour before thoroughly washing to remove unbound bacteria. As expected, the ICAM-targeted bacteria attached in significantly higher amounts than the unmodified control (FIGS. 2D and 2E). To confirm that EC attachment was due to successful presentation of streptavidin-functionalized aICAM-1 on biotin-modified bacteria, a panel of controls were analyzed. Biotinylated and unmodified bacteria were pre-incubated with aICAM-1 or the aICAM-streptavidin conjugate to evaluate whether surface conjugation, as opposed to passive adsorption, was required to provide targeted functionality. Controls demonstrate that surface functionalization with biotin and subsequent attachment of the aICAM-streptavidin conjugate is required for sufficient antibody display and improved bacterial attachment to Caco-2 cells (FIG. 2D).

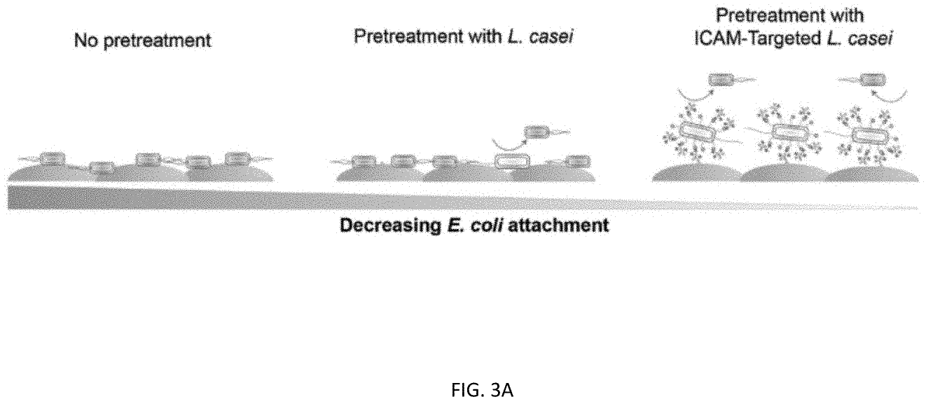

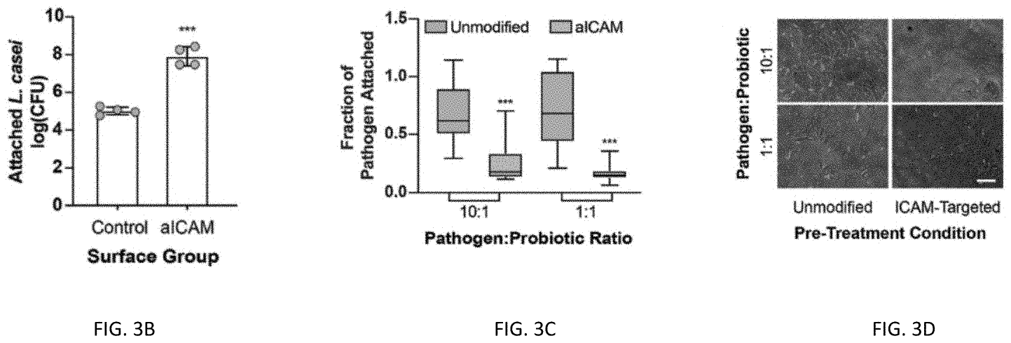

A known beneficial and microbiome-regulatory function of commensal bacteria is the prevention of pathogen attachment and colonization in the GI tract. It was determined whether compositions and systems described herein could be used as an anti-adhesion therapy by preventing a model pathogen from attaching to epithelial cells (FIG. 3A). For this study, the common dairy probiotic species LC was used, which has anti-inflammatory and anti-virulence properties. Furthermore, Lactobacillus species have been shown to mediate pathogen attachment by forming a steric barrier on mammalian cells or the mucosal lining. It was hypothesized that this mechanism could be enhanced by the addition of synthetic adhesins targeted to Caco-2 cells. By targeting LC to ICAM-1, LC adherence to Caco-2 cells is significantly increased compared to an unmodified control (FIG. 3B), determined by plating and enumerating viable CFUs following the removal of Caco-2 monolayer.

It next analyzed whether prophylactic treatment of Caco-2 cells with LC can reduce subsequent attachment of a bacterial pathogen. Common pathogenic bacterial species show significant toxicity towards mammalian cells, leading to compromised integrity of the Caco-2 monolayer. For our in vitro model, it was found that this toxicity limited the use of standard quantitative analysis methods due to the compromised monolayer, leading to high rates of pathogen attachment to the polystyrene well plate. To maintain Caco-2 monolayer integrity and accurately quantify bacterial attachment to Caco-2 cells, a GFP-expressing EC DH5α strain was selected as the model pathogen. Caco-2 cells were treated with either unmodified or ICAM-targeted LC for 1 hour. After washing the cells to remove unbound LC, Caco-2 cells were challenged for 1 hour with either an equal (1:1) or 10-fold higher (10:1) ratio of pathogen to probiotic (FIG. 3C). ICAM-targeted LC was 3-fold more effective than the unmodified control in preventing EC attachment to Caco-2 cells (FIG. 3C). Interestingly, the efficacy of ICAM-targeted LC was independent of the pathogen:probiotic ratio, highlighting how a small population of targeted probiotics can be used to limit attachment of a pathogen, even when the pathogen is present at an order of magnitude higher concentration. Representative images demonstrate the reduction in EC attachment following treatment with ICAM-targeted LC (FIG. 3D). The dramatic reduction in EC attachment to live Caco-2 cells demonstrates that synthetic adhesins can be used to create a barrier against pathogen attachment by granting the probiotic an adherence advantage.

To investigate the benefit of targeting a general receptor on Caco-2 cells during a competitive challenge model, LC and EC were incubated simultaneously on Caco-2 cells. Following washes to remove unbound bacteria, it was found that surface modification does not significantly affect the attachment of EC compared to unmodified LC. It is believed that this is because the system relies on physically excluding pathogens after LTB binding, as opposed to directly competing with the pathogen for specific adhesin receptors. As such, targeting enhances the ability for LC to form a physical steric barrier that improves pathogen exclusion only in a prophylactic model. Modification of the LC surface with antibodies directed towards EC binding sites would likely provide a direct competitive advantage to LC, as previous reports of genetically engineered probiotics that directly compete with pathogen binding can reduce and displace bound pathogen.

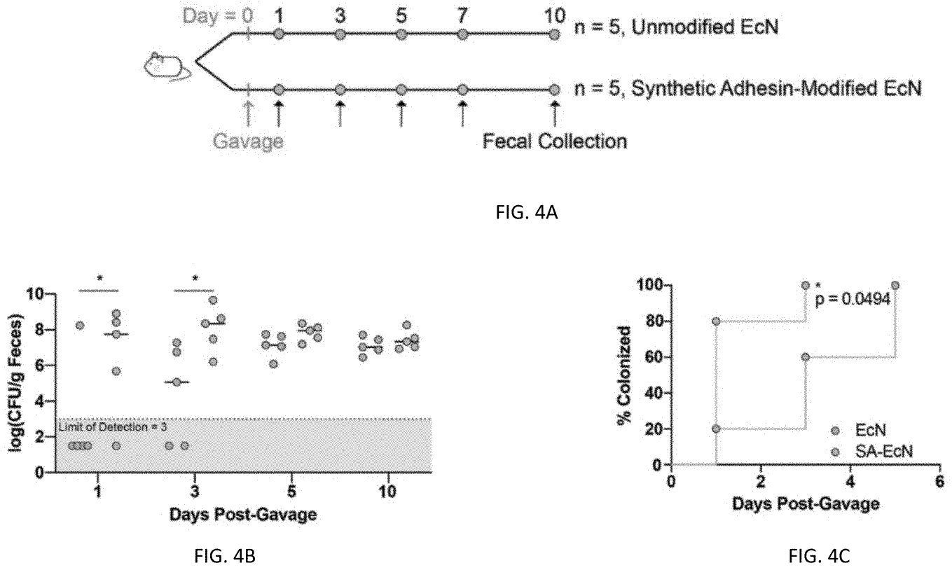

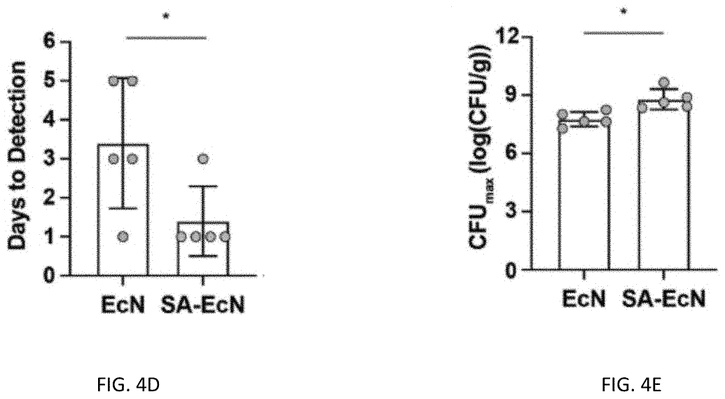

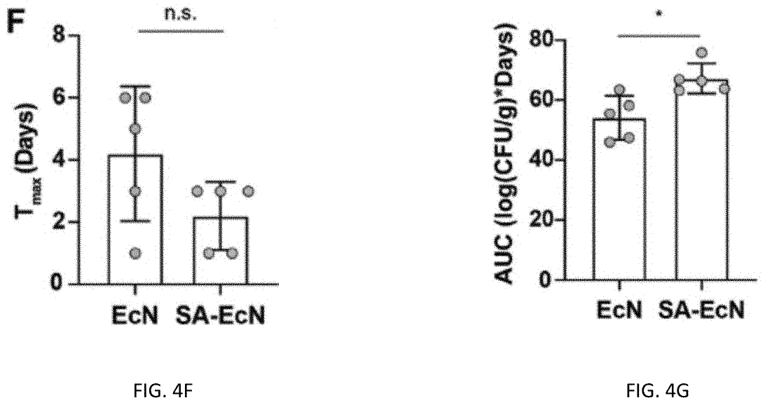

Due the importance of surface adhesins in the colonization of biotherapeutics in the GI tract, the effect of synthetic surface modifications on in vivo colonization was investigated. E. coli Nissle 1917 (EcN), a probiotic with extensive clinical and preclinical data that naturally colonizes the GI tract was used for in vivo studies. To enhance the adherence of EcN to the GI tract, anti-MUC2 antibodies (aMUC2) were attached to the surface as previously described. To reduce the cost of the platform and improve its potential for translation, a polyclonal antibody was selected for in vivo studies in contrast to the monoclonal anti-ICAM-1 antibodies used for in vitro studies. MUC2 is an essential component of intestinal mucus, a common adhesin target for bacteria, and a mediator of host-bacterial interactions at the mucosal interface, making it a ubiquitous and bio-inspired choice for a synthetic adhesin (SA). Prior to administration of EcN, mice were pre-treated with streptomycin for 24-hours, followed by an 18-hour washout period of the antibiotic. Antibiotic pre-treatment is routinely used to enable LTB colonization in clinical settings, including for FMTs and LTB consortia. Streptomycin specifically opens a niche for EcN colonization by selectively removing facultative anaerobes, leaving the abundance and diversity of remaining anaerobes intact. In the model described herein, it was found that EcN fails to colonize mice in the absence of either antibiotic treatment or a wash-out period. Unmodified and aMUC2 synthetic adhesin-modified (SA-EcN) EcN were delivered to mice via oral gavage and colonization was tracked over a period of 10 days (FIG. 4A). Fecal pellets were used to quantify the intestinal EcN concentration, as fecal bacterial concentration has previously been used as a proxy for bacterial load in the intestines. Mice treated with SA-EcN had significantly higher bacteria in their feces on days 1 and 3 following gavage (FIG. 4B) and both groups stabilized to approximately 10⁷ CFU g⁻¹ feces by day 5. Defining colonization as the presence of detectable bacteria in the feces, the length of time required for all mice in a group to become colonized was analyzed (FIGS. 4C and 4D). Synthetic adhesins significantly reduced the time required to reach 100% colonization, with all mice in the SA-EcN treatment group having detectable EcN in their feces by day 3.

To understand the effects of earlier colonization via synthetic adhesins on microbe-host interactions, pharmacokinetic parameters (FIGS. 4E-4G) that are traditionally used to understand the absorption and elimination of a therapeutic were calculated. Previous work for live biotherapeutics has used pharmacokinetics to describe LTB colonization, or the effect that LTBs have on diagnostic read-outs and co-administered therapeutics. However, to our knowledge, no previous work has applied traditional pharmacokinetics to describe the benefits of a rationally designed delivery system for LTBs. The results presented herein show that SA-EcN reached a significantly higher viable concentration (CFUₘₐₓ) than unmodified EcN (FIG. 4E). Additionally, the time at which the CFUₘₐₓ occurs (tₘₐₓ) is lower for SA-EcN (FIG. 4F). Therefore, synthetic adhesins enable EcN to rapidly reach a high concentration in the GI tract. To determine the long-term consequences of the tₘₐₓ and Cₘₐₓ, the area under the curve (AUC) was calculated for both SA-EcN and the unmodified control. The AUC is the integral for the plot of EcN concentration in feces vs. time (FIG. 4B) and is a measure of the total exposure to a therapeutic. The AUC of SA-EcN was significantly higher than the unmodified control (FIG. 4G). Therefore, even though synthetic adhesins do not lead to a long-term increase in colonization, their advantages at early timepoints increase an animal's total exposure to EcN by 20%. For biotherapeutics that secrete small molecules or biologics, this would lead to a direct increase in the patient's exposure to their bioactive compounds.

The effect of synthetic adhesins is likely transient for two reasons: (i) surface modifications dilute as bacteria proliferate in vivo and (ii) mice are coprophagic. This system relies on chemical conjugation to the surface of bacteria, which will lead to dilution of the conjugated targeting ligands on the LTB surface as they grow. Therefore, as the bacteria grow in vivo, they lose their synthetic adhesins and, subsequently, their ability to specifically adhere to their synthetic adhesin's target. While the dilution of surface modifications on the LTB surface may be a limitation of the platform, it also represents an advantage compared to permanent alterations of the LTBs that may introduce safety concerns of administering genetically engineered bacteria or can interfere with the natural mechanism of action for the LTB. Furthermore, the in vivo data collectively demonstrate that the early advantages provided by antibody targeting of LTBs is sufficient to establish an intestinal niche, enabling them to proliferate in the GI tract and withstand clearance mechanisms such as peristalsis and mucosal clearance. In addition to the dilution of targeting ligands, the mice are not individually housed and therefore will ingest feces throughout the study, re-inoculating their intestinal tract with shed EcN. These two processes will saturate and stabilize the amount of EcN in the intestinal tract, as shown starting at day 5 (FIG. 4B). Because coprophagy is unique to rodents, the benefits of synthetic adhesins may be understated by this data.

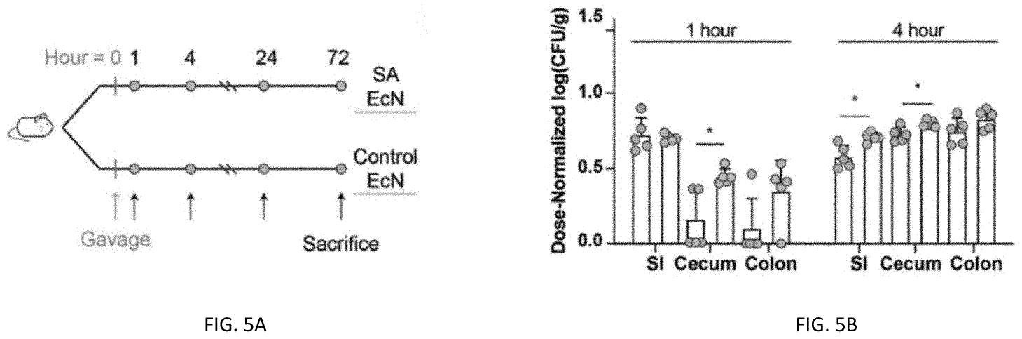

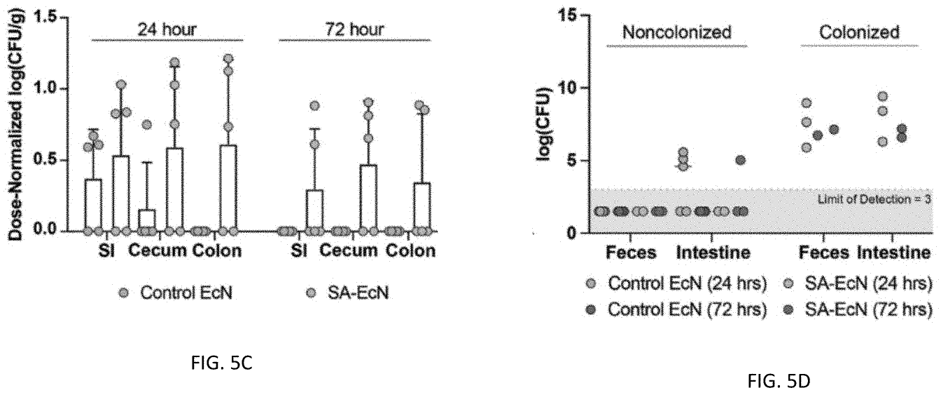

To investigate the effect of synthetic adhesins on the short-term transit of EcN in the intestinal tract, mice were gavaged with either SA-EcN or an unmodified control and sacrificed 1- and 4-hours later (FIG. 5A). To confer bioluminescence and image EcN on an In Vivo Imaging System (IVIS), a strain bearing no native plasmids was transformed with the pGEN-luxCDABE plasmid. The intestinal tracts were harvested and imaged using IVIS to visualize distribution of the bacteria along the GI tract, which showed EcN in the small intestine at 1-hour post-gavage and all segments of the GI tract by 4-hours post-gavage. The small intestine, cecum and colon were homogenized and plated to determine the viable abundance of EcN in each organ (FIG. 5B). The plating data proved to be a more sensitive method for detecting and quantifying EcN in the intestinal tract, revealing that mice treated with SA-EcN have a significantly higher abundance of EcN in their cecum at 1-hour, indicating faster transit than unmodified EcN. By 4 hours post-gavage, mice treated with SA-EcN have significantly higher viable bacteria in their small intestines and ceca. Synthetic adhesins therefore alter the transit of EcN in the GI tract, enabling a population of SA-EcN to reach the cecum faster (FIG. 5B, left), while remaining EcN have an increased residence time in the small intestine. Additionally, SA-EcN appear to persist in the cecum at a higher abundance than the unmodified control (FIG. 5B, right). This strongly supports the colonization data by highlighting that modification with synthetic adhesins results in both faster appearance and higher viable amounts of EcN in the feces of treated mice.

To confirm that the increased abundance of EcN in the feces of SA-EcN treated mice at early timepoints (FIG. 4B) is an indicator of improved intestinal colonization rather than rapid transit and clearance, intestinal colonization was assessed as described above at 24- and 72-hours post-gavage. At 24-hours, 60% of the SA-EcN mice were colonized in all segments of their intestinal tract, including the small intestine, cecum, and colon (FIG. 5C, left). While three control mice had detectable EcN in their small intestine, none were colonized throughout their GI tract and by 72-hours, only SA-EcN treated mice had viable EcN in the intestinal tract (FIG. 5C, right). The intestinal tracts were additionally imaged using IVIS, which showed EcN in the intestinal tract of mice in both groups at 24-hours, but only SA-EcN treated mice by 72-hours. Importantly, none of the mice in the control group had viable EcN in their feces at either 24- or 72-hours post-gavage. To determine the relationship between fecal and intestinal samples, the abundance of EcN in the feces and intestinal tracts in two groups of mice were correlated: those with detectable EcN in their feces (colonized) and those without (noncolonized) (FIG. 5D). It was found that colonized mice had comparable levels of EcN in their feces and intestinal tracts (FIG. 5D, right), while noncolonized mice showed lower or no viable EcN in their intestinal tracts (FIG. 5D, left). From this data, it was concluded that the presence of EcN in feces is indeed indicative of intestinal EcN colonization.

Taken together, the data presented herein demonstrates that in all cases where fecal counts are detectable, the intestinal tract is colonized with a comparable level of EcN (FIG. 5D). From this, it is clear that treatment with SA-EcN leads to higher abundance in the small intestine and cecum immediately following administration (FIG. 5B), enabling improved intestinal and fecal colonization in the first three days (FIG. 4B, 5C). Therefore, it was hypothesized that synthetic adhesins improve the ability of EcN to rapidly form an intestinal niche that acts as a stable depot to sustain shedding of excess EcN into the feces. This agrees with literature on probiotic and commensal species, where the fecal microbiome is frequently used as a proxy for the intestinal environment, as well as known mechanisms of pathogen colonization, where formation of an intestinal niche supports a sustained intestinal population that is responsible for fecal shedding. Finally, this hypothesis is further supported by the fact that all mice in the long-term colonization study were stably colonized with EcN at least a month following treatment, suggesting an equilibrium between EcN growth and fecal shedding during this time.

This example demonstrates a rapid and modular platform that can be used with any given bacteria and antibody combination to modify the bacterial surface, including over-the-counter probiotics, beneficial consortia, and LTBs used in the clinic. It has been shown that surface modification improves LTB adhesion, enhancing the ability to exclude pathogenic bacteria in vitro, even in the presence of a 10-fold higher pathogen burden. Additionally, this example presents a new perspective on LTB pharmacokinetic analysis, providing a framework for designing and evaluating engineered drug delivery systems for LTBs. Using this analysis, it was demonstrated that synthetic adhesins enable an early colonization advantage that supports an intestinal LTB depot, leading to an increase in their maximum concentration and the total exposure to the biotherapeutic over time without impeding subsequent LTB growth in, or interaction with, the GI tract. For LTBs engineered to secrete biotherapeutics or for those that are active for only a short window following administration, such as Synlogic's Phase I/II candidate SYNB1618, this early advantage in colonization and proliferation in the intestinal tract will directly correlate with improved patient exposure to the biotherapeutic and efficacy of the LTB. Notably, the principles described in this example can be extrapolated to other bacterial and/or microbial species.

### Materials and Methods

Cell Lines and Culture. Caco-2 (ATCC HTB 37) cells were purchased from the University of North Carolina at Chapel Hill Tissue Culture Facility. Caco-2 cells were cultured in DMEM media supplemented with 1% penicillin-streptomycin and 10% Fetal Bovine Serum (FBS). Lactobacillus casei (ATCC 393) and Bacillus coagulans (ATCC 7050) were purchased from ATCC. Escherichia coli DH5α was purchased transformed with a pBS-ldhGFP plasmid, a gift from Michela Lizier (Addgene plasmid #27170; http://n2t.net/addgene:27170; RRID:Addgene_27170).^{[18],[34]} Escherichia coli Nissle 1917 was a gift from Nathan Crook and was transformed with the pGENlux-CDABE plasmid, a gift from Harry Mobley (Addgene plasmid #44918; http://n2t.net/addgene:44918; RRID: Addgene_44918).^{[27]} All bacterial cultures were inoculated from glycerol stocks 24 hours before use in a study. L. casei (LC) was grown in a static incubator at 37° C. in MRS media, while E. coli (EC), B. coagulans (BC), Pseudomonas aeruginosa (PA), and Salmonella typhimurium (ST) were grown in a shaking incubator (200 rpm) at 37° C. in Lysogeny Broth (LB) or Nutrient Broth (NB), respectively.

Biotinylation of Bacterial Surface. Bacteria cultures were inoculated from a single colony and incubated overnight before use. Bacteria was harvested via centrifugation for 10 minutes at 4,000 rpm and washed three times with sterile, ice-cold Phosphate Buffered Saline (PBS). Biotinylation was conducted with sulfo-NHS-functionalized biotin (EZ-Link Sulfo-NHS-Biotin, ThermoFisher) with 1 mg of sulfo-NHS-biotin per mL of liquid bacteria culture. All biotinylation reactions were conducted with bacteria at an OD600 of 1.0. The reaction proceeded on ice for 20 minutes. Following biotinylation, bacteria were harvested via centrifugation and washed three times with ice-cold sterile PBS, as previously described. Prior to biotinylation of Visbiome® surface, a single Visbiome® capsule was dissolved in PBS and washed 2× in PBS to remove capsule contents.

Biotinylated Bacteria Attachment to Streptavidin. To confirm biotinylation, all biotinylated species were incubated with a 1:100 dilution of a fluorescent streptavidin conjugate (Alexa Fluor® 568 Streptavidin; ThermoFisher). Bacteria were examined and imaged using an epi-fluorescence microscope (Revolve; Echo). Biotinylated EC were incubated on a streptavidin-coated plate with serial dilutions starting at OD=0.5. Bacteria were incubated with constant agitation (200 rpm) for 1 hr, washed four times with sterile PBS, and fluorescence was quantified using a microplate reader (Synergy H1; BioTek).

Antibody Attachment to Biotinylated Bacteria. Antibody conjugates were formed using a commercially available streptavidin conjugation kit (Abcam) according to the manufacturer's instructions with an R6.5 anti-ICAM-1 monoclonal antibody (ThermoFisher Scientific #BMS1011). Antibody conjugates were confirmed via protein gel electrophoresis. A total of 1 μg of native protein was loaded in a TGX Stain-Free™Precast Gel and run according to manufacturer's instructions (Mini-PROTEAN®; Bio-Rad). Bands were compared to Precision Plus Protein™ unstained protein standards. Following confirmation of successful conjugation, antibody conjugates were incubated with biotinylated bacteria for 20 min with continuous agitation. Bacteria were harvested via centrifugation and washed three times as described previously to remove unbound antibody conjugates.

Bacterial Attachment to Caco-2 Cells. Caco-2 cells were seeded in tissue culture treated 96-well plates with a cell density of 1×10⁵ cells mL⁻¹ and grown to confluence. Prior to the attachment study, Caco-2 cells were washed twice with pre-warmed unsupplemented media to remove FBS and pen-strep. Bacteria that were incubated with antibodies were prepared as previously described. Following the final wash, bacteria were resuspended in unsupplemented DMEM. 100 μL of 3.5×10⁹ cells mL⁻¹ (OD600=0.5) EC were prepared by dilution in DMEM and incubated with Caco-2 cells for 1 hr at 37° C. Caco-2 cells were washed four times with pre-warmed Hanks Balanced Salt Solution (HBSS) and fluorescence was quantified using a microplate reader (Synergy H1; BioTek). Unfixed cells were immediately examined and imagined under a hybrid epi-fluorescence microscope (Revolve; Echo).

Competitive Exclusion Studies. To assess bacterial toxicity towards Caco-2 cells and the effect on the Caco-2 monolayer, cultures of EC, ST, and PA were incubated with Caco-2 monolayers for 1-hour and washed 3× to remove unbound bacteria. Monolayer damage was assessed using microscopy on an epi-fluorescence microscope (Revolve; Echo). For exclusion studies, LC was cultured, biotinylated, and coated with ICAM-1 antibody as previously described. To confirm LC attachment to Caco-2 cells, unmodified, biotinylated and ICAM-1-targeted LC were incubated on a Caco-2 monolayer for 1-hr. Cells were washed to remove unbound LC, trypsinized to remove Caco-2 cells, and plated to enumerate viable, adhered LC. For the exclusion study, both LC and EC cultures were suspended in unsupplemented DMEM. Confluent Caco-2 monolayers were used for the competitive exclusion studies and were washed twice with unsupplemented DMEM prior to the study. A pilot study was conducted to determine the appropriate concentrations of EC and LC. Unmodified LC (OD=1.0) was mixed with varying concentrations of EC (OD=0.1 to 1.0). 100 μL of the bacteria mixture was incubated with Caco-2 cells for 1 hr and washed four times with pre-warmed HBSS. Fluorescence was quantified on a plate reader, as previously described. Optimal conditions (EC at OD=0.4, LC at OD=1.0) were selected, and competition studies were repeated using both unmodified LC and antibody-decorated LC.

Mouse Colonization Studies. Animal studies were conducted in accordance with and approved by the Institutional Animal Care and Use Committee (IACUC) of The University of North Carolina at Chapel Hill. 8-week old female BALB/c mice were used for in vivo colonization studies. Mice were purchased from Charles River Labs (Stock #028) and acclimated for at least 72 hours prior to use. Streptomycin was administered to mice ad lib in the drinking water for 24 hours (5 g L⁻¹). Mice were placed back on an automatic watering system for 18 hours prior to administration of bacteria. E. coli Nissle 1917 (EcN) with a genomically integrated GFP gene and a plasmid conferring kanamycin resistance was used for in vivo mouse colonization studies. EcN was grown overnight to saturated conditions, washed to remove media, and biotinylated as described above. An anti-MUC2 antibody (Abcam #ab76774) was conjugated to streptavidin as described above and attached to biotinylated EcN. 100 μL of bacterial culture (unmodified or anti-MUC2 modified) was administered via oral gavage to mice (n=5 per cage) in sterile normal saline solution. Feces was collected starting at Day 1 by placing mice in a sterile, empty cage and waiting for approximately 2-5 pellets of bacteria to pass. Pellets were weighed and sterile Phosphate Buffered Saline was used to homogenize the pellets. Serial dilutions of the EcN was plated on selective kanamycin (50 μg mL^{31 1}) plates. Colony forming units were enumerating after 72 hours of growth at 37° C.

Distribution and Abundance of EcN. 8-week old female BALB/c mice were purchased from Charles River Labs (Stock #028) and acclimated for at least 72 hours prior to use. Streptomycin was administered to mice and unmodified and anti-MUC2-targeted EcN were prepared for oral gavage as described above. A bioluminescent strain of EcN was used for in vivo distribution studies to visualize the bacterial transit in the GI tract. EcN bearing no native plasmids was transformed with pGEN-luxCDABE.^{[34]} 100 μL of bacterial culture was administered via oral gavage to mice (n=5 per cage) in sterile normal saline solution. Mice were sacrificed 1- or 4-, 24- or 72-hours post-gavage and the intestinal tracts were harvested, imaged with an In Vivo Imaging System (IVIS) Kinetics Optical System (PerkinElmer, CA), and segmented into the small intestine, cecum and colon. Fecal samples were collected from mice in the 24- and 72-hour cohorts at 6-, 12-, 18-, 24-, 48-, and 72-hours post-gavage and processed as described above. All intestinal segments were homogenized twice with an MP Biomedical FastPrep-24 homogenizer using 1.4 mm ceramic bead-filled tubes (15 seconds, 6.5 M s⁻¹). Intestinal samples were serially diluted and plated on ampicillin (100 μg mL⁻¹) selective LB agar plates. Viable colony forming units were enumerated and data was normalized to the dose given to each animal. All IVIS images were scaled to visualize the lowest signal in each image.

### EXAMPLE 2

### Orally Administered Live Biotherapeutic Products (LBPs)

In Example 1 above, it was demonstrated that N-hydroxysulfosuccinimide ester-based (sulfo-NHS ester) chemistry can be used to present targeting ligands on the LBP surface to improve their attachment to specific proteins on abiotic surfaces, mammalian cells, and the murine GI tract. This platform has a number of advantages, including a rapid reaction time (<20 minutes), compatibility with any targeting ligand with an accessible carboxyl or amine group, and modularity across bacterial species due to the use of ubiquitous primary amines for the bioconjugation reaction. In the present example, using the platform of Example 1, it is demonstrated that there are optimal LBP concentrations and residence times that maximize the attachment of modified LBPs to their target proteins, while LBP growth dilutes the surface modification and decreases their attachment to target proteins. Additionally, it shown that this platform does not interfere with therapeutically essential LBP functions, including their ability to survive and grow during standard batch culture, metabolize key therapeutic molecules, and colonize the murine GI tract (in this example, we show this for a non-targeted surface modification whereas in Example 1 we demonstrated that a targeted surface modification improves colonization of the murine GI tract). Finally, it is demonstrated that target binding is conserved for up to one week following storage under clinically relevant conditions, supporting the clinical potential of surface modifications as an LBP delivery system. Collectively, this work supports the use of LBP bioconjugation as a delivery strategy, while simultaneously establishing an experimental pipeline for the characterization of LBP delivery systems for oral delivery.

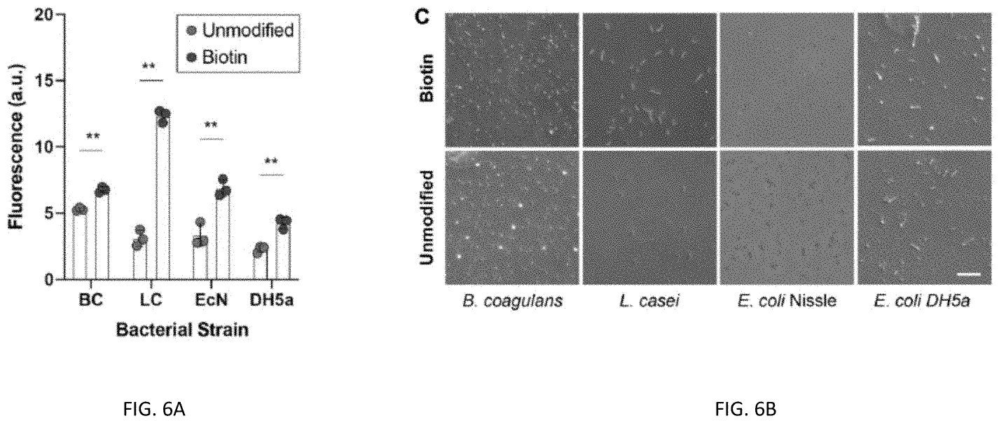

Initially, the modularity of surface modifications across was demonstrated across LBP species. Escherichia coli Nissle 1917 (EcN), E. coli DH5α, Lactobacillus casei, and Bacillus coagulans were modified using sulfo-NHS-based chemistry. Sulfo-NHS ester functionalized moieties react with amine groups on the surface of LBPs, forming an amide bond between the LBP surface and the functionalized group (FIG. 1A). Importantly, sulfo-NHS ester-based chemistry can be used with a wide variety of targeting ligands, making it a modular chemical approach towards modifying the LBP surface. As a model targeting ligand, functionalized biotin (sulfo-NHS-biotin) was conjugated to the surface of multiple LBP strains. To confirm the conjugation and functional presentation of biotin, unmodified and biotinylated LBPs were incubated with a fluorescent streptavidin probe. Following incubation, fluorescent signal significantly increased for all strains tested (FIG. 6A). The extent of biotinylation, observed by the intensity of the fluorescent signal, differed between strains, which is consistent with previous reports of this system. The binding of fluorescent streptavidin on the LBP surface was visualized using fluorescence microscopy (FIG. 6B), confirming that streptavidin specifically binds to the surface of biotinylated, but not unmodified, LBPs.

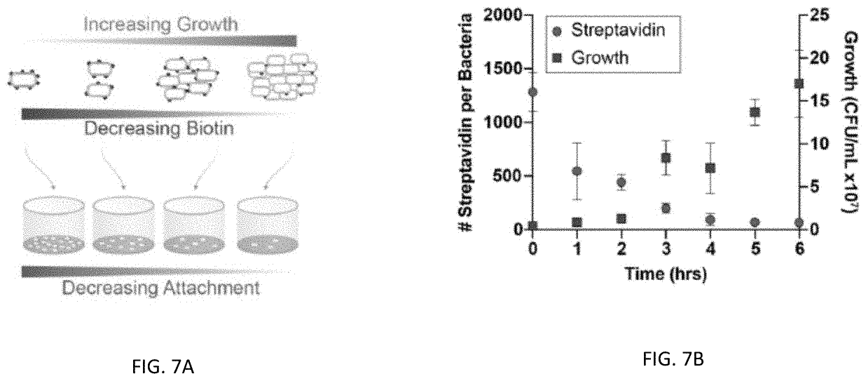

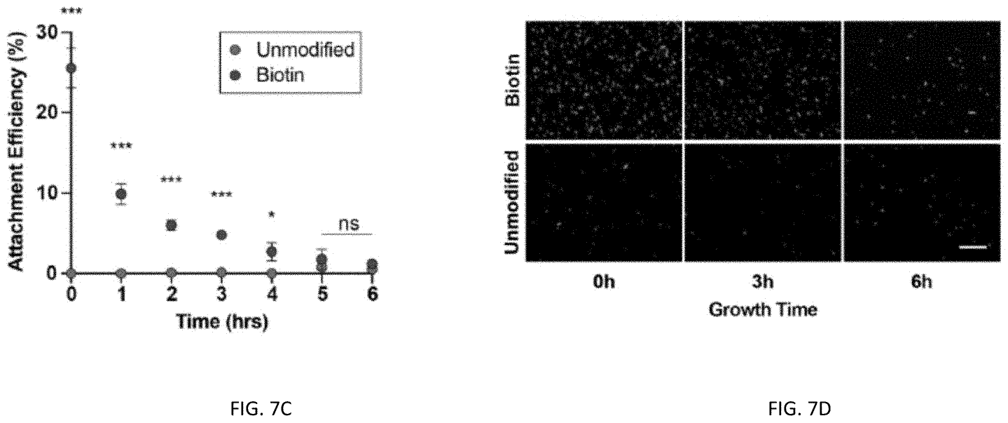

In Example 1 herein, it was shown that surface modification can increase LBP attachment to specific protein targets, including those on abiotic and biotic surfaces. However, because this platform relies on chemical conjugation, the modification will dilute as LBPs grow, potentially impacting target attachment. While the transient nature of the modification ensures the LBP is reverted to its initial form, lowering its safety risk relative to permanent genetic alterations, the dynamics of biotin loss can render insufficient target binding. To better understand the kinetics of modification dilution, the surface of a GFP-expressing strain of E. coli DH5α was modified with biotin. As shown in FIG. 7A, the loss of biotin on the LBP surface during growth was quantified and its effect on the attachment of LBPs to a streptavidin-coated well-plate. Biotin concentration on the LBP surface was measured using a fluorescent streptavidin probe, normalized to the number of colony forming units (CFU) in a sample. It was found that the dilution of surface biotin correlates with the exponential growth of the modified LBP (FIG. 7B). At each timepoint, unmodified and biotinylated LBPs were incubated on a streptavidin-coated plate for 20 minutes and calculated the attachment efficiency, measured as the percent of fluorescent signal retained after washes to remove unbound LBPs. It was found that attachment decreases as a consequence of biotin dilution (FIG. 7C), which is supported by fluorescent microscopy of the well-plate floor (FIG. 7D). Additionally, it was discovered that a minimum concentration of 50 surface biotin molecules per bacteria is required to achieve increased attachment relative to the unmodified control and, notably, biotinylation provides an attachment advantage for four hours during exponential batch culture, as seen in FIG. 7C.

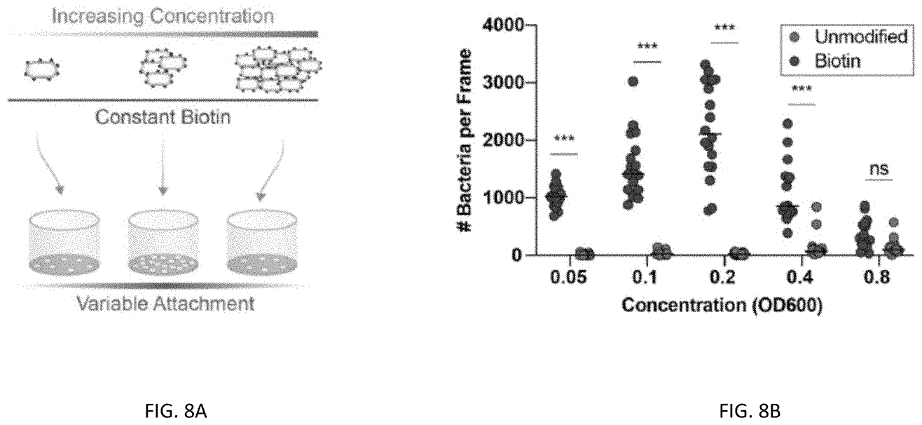

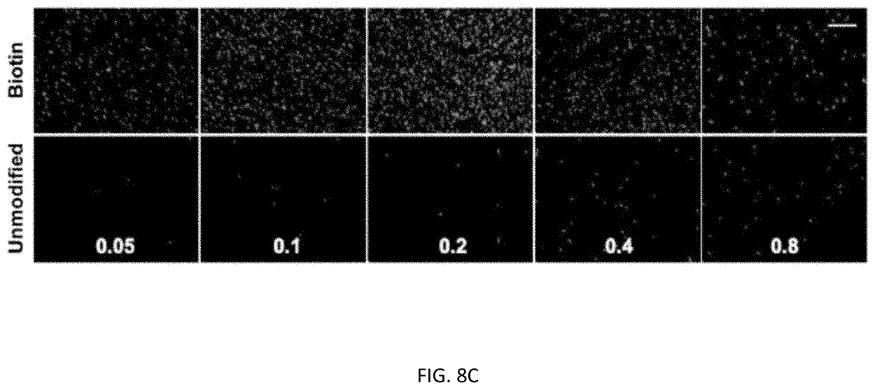

The effect of concentration on the attachment of biotinylated and unmodified LBPs to target proteins was assessed next, a key parameter for determining an effective dose following modification. Varying concentrations of biotinylated or unmodified E. coli DH5α were incubated on a streptavidin-coated well-plate for 20 minutes, washed thoroughly (FIG. 8A), and LBP attachment was quantified using fluorescence microscopy (FIGS. 8B and 8C). It is expected that LBP attachment to an abiotic surface is proportional to concentration, which was observed in the unmodified control. However, it was found that while the attachment of biotinylated LBPs increases with concentration in relatively dilute samples, attachment is inhibited at high concentrations and a clear attachment maximum exists (OD=0.2, FIG. 8B). it was found that while the specific concentration associated with maximum attachment is dependent on study conditions, there is a negative correlation between attachment efficiency and concentration that is conserved across study conditions. The inhibition is possibly the result of biotin on the LBP surface becoming sterically hindered as the LBP concentration increases without a corresponding increase to surface area or streptavidin. Therefore, the competition for available streptavidin binding sites will increase and fewer LBPs will reach a sufficient threshold of interactions with their target to maintain attachment following washing.

Next, analysis was conducted regarding how an initial attachment advantage benefitted biotinylated LBPs during growth. Following attachment in FIG. 8B, LBPs were incubated at 37° C. to allow for growth in one-hour increments and then washed to mimic the dynamic conditions of the GI microenvironment. It was found that the attachment of biotinylated LBPs decreased with time for all concentrations, further supporting that biotin loss on the LBP surface during growth negatively influences attachment, as shown in FIG. 7B. Furthermore, biotinylated LBPs retained an attachment advantage over the unmodified control for 5-hours when they are attached prior to growth.

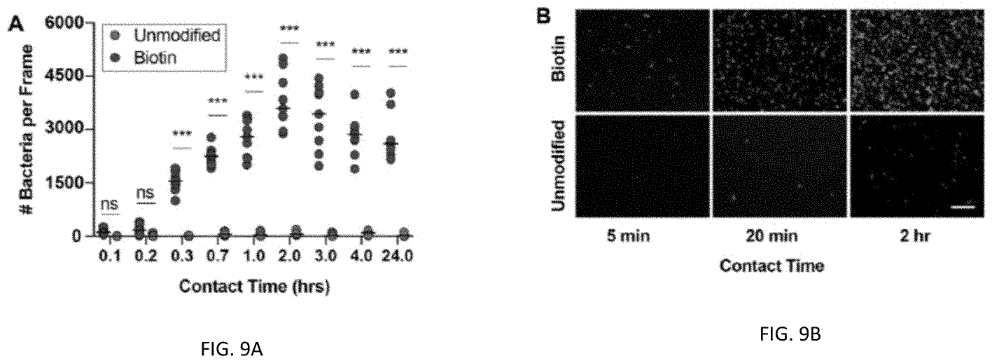

The effect of residence time on the attachment of modified LBPs to their target protein was analyzed. Attachment has been shown to increase with residence time (i.e. the contact time between the microbe and its target) due to increased collisions with the attachment surface. Understanding the residence time required to enable sufficient attachment of LBPs to the GI tract is important for rationally designing oral delivery systems, as attachment is a critical step in the colonization of LBPs.²⁵ For this study, E. coli DH5α was incubated at a constant concentration (OD=0.25) for varying lengths of time (5 minutes to 24 hours) on a streptavidin-coated well-plate at 4° C. to limit growth and viability loss. At each timepoint, the well-plate was washed and LBP attachment was quantified using fluorescent microscopy (FIGS. 9A and 9B). Results were consistent with previously published studies, demonstrating that LBP attachment increases with residence time until saturation is reached, which occurs after approximately 2 hours. Compared to optimizing the LBP concentration alone, we were able to nearly double the attachment of biotinylated LBPs by extending the residence time (˜2100 bacteria per frame in FIG. 8B vs. ˜3800 in FIG. 9A).

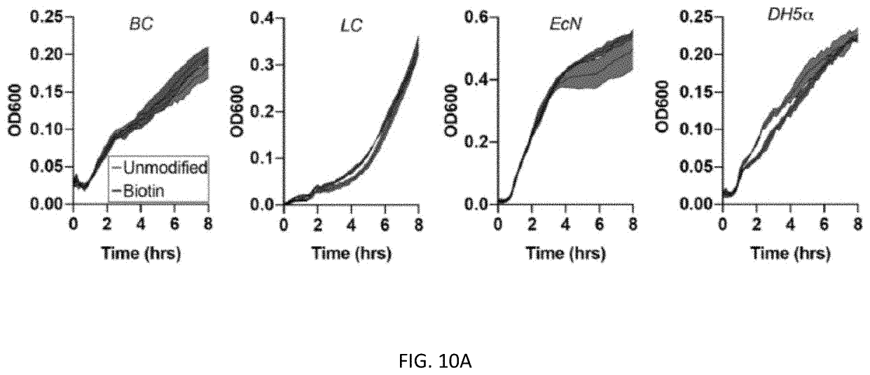

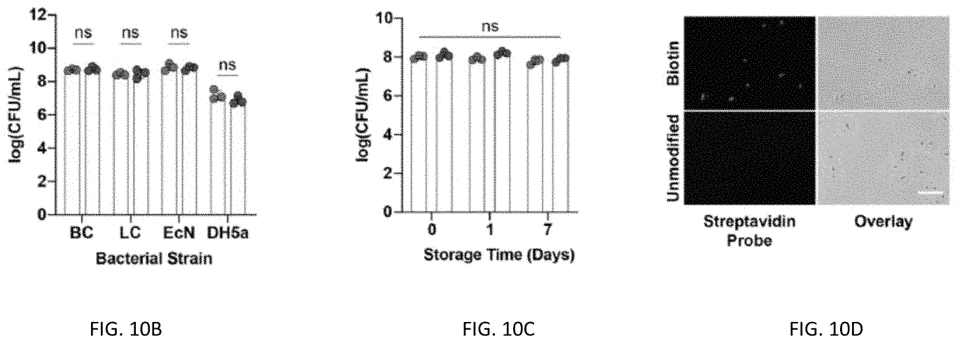

Current clinical use of LBPs, including fecal microbiota transplants and donor-derived spore-based therapeutics, rely on defined processing steps that attempt to maximize viability and are compatible with cryopreservation. Therefore, the effects of surface modification on the growth, viability, and cryopreservation of LBPs were characterized. Biotinylation did not affect the growth of any LBP strain tested during an 8-hour period (FIG. 10A), nor did biotinylation significantly alter LBP viability, measured as colony forming units (CFU) (FIG. 10B). Clinically, preservation of LBP formulations is essential for their practicality, as they can rarely be used immediately upon preparation and they may require transport between manufacturing facilities and clinics. As such, EcN was tested for its storage under common cryopreservation conditions (25% glycerol, −80° C.). Surface modification did not significantly alter LBP viability for up to one week of storage (FIG. 10C) and the functionality of surface modification proved to be compatible with cold storage, as streptavidin binding was preserved at each timepoint tested (FIG. 10D). This presents a key advantage for this modification platform, as it improves the potential that LBP formulations can be prepared and modified at scale, prior to quality control, packaging and storage processing steps. Additionally, the ability to modify LBPs prior to storage and shipping may allow for an off-the-shelf therapeutic that alleviates the need to conduct post-preservation modifications of the LBP at the point-of-care, which can be burdensome for patients, clinics or hospitals.

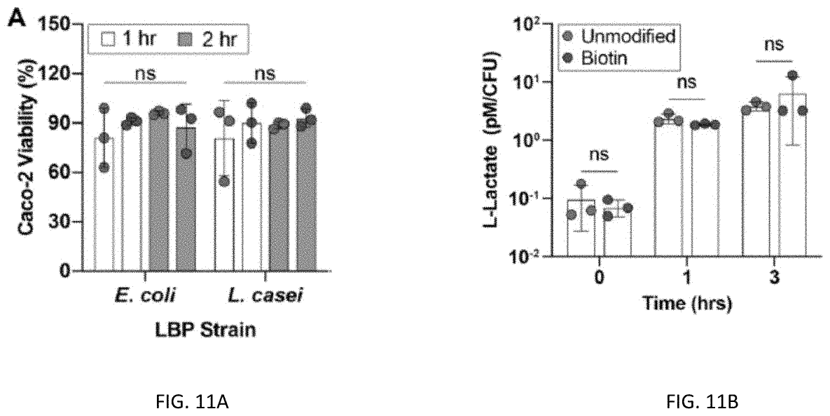

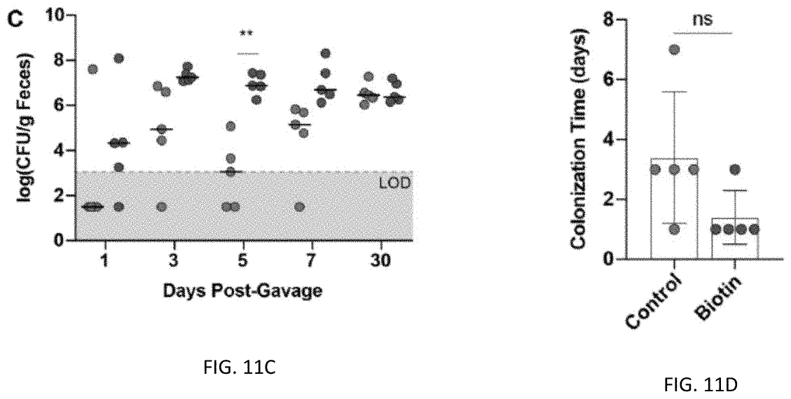

As LBPs act through multiple mechanisms, the ability to effectively access and use nutrient sources (metabolism) in order to survive and proliferate within the intestinal tract (colonization) without significantly influencing the health of their human host (mammalian toxicity) is essential for their therapeutic efficacy. While it was confirmed that biotinylation does not inhibit LBP growth or viability, it is not clear if surface modifications alter these more complex bacterial functions. To determine the influence of surface modifications on these parameters, the viability of mammalian cells after exposure to the candidate LBPs EcN and L. casei was first evaluated using an MTT assay. LBPs were incubated on Caco-2 cells, a colorectal cancer cell line commonly used as a model of the intestinal epithelium, at concentrations ranging from 10⁶ to 10⁸ CFU/well for up to two hours. No significant toxicity against mammalian cells for either LBP strain following biotinylation at 10⁶ or 10⁷ CFU/well (FIG. 11A) was found. However, at high LBP concentrations (10⁸ CFU/well), it was found that EcN and L. casei contributed to the reduction of MTT bromide to its formazan, resulting in signals above the positive control and decreasing the reliability of results.

Next, the impact of biotinylation on the metabolism of L. casei, which produces lactic acid through the fermentation of glucose, was tested. The metabolic byproduct of lactic acid has been shown to mediate diverse disease states, including NSAID-induced small intestine injury, diabetes complications, and pathogen infections. As such, it is essential that surface modifications of L. casei do not interfere with lactic acid secretion. Lactic acid production under two conditions was measured: during growth in MRS media and during fermentation in minimal media supplemented with glucose to inhibit growth and ensure that biotin dilution did not influence results. It was found that biotinylation does not significantly alter lactic acid production under either growth condition.

Example 1 demonstrated that modification of the LBP surface with targeting ligands directed against in vivo targets significantly improves their short-term adhesion in the GI tract, enabling them to quickly establish an intestinal niche and improving their pharmacokinetics. While targeting specific receptors in the GI tract appears to improve colonization, little is known about the influence of surface modifications more broadly on the interactions between LBPs and the GI environment. Indeed, alternative approaches to modifying LBP surfaces, such as encapsulation, can physically impede LBP growth or interactions with the GI environment. Therefore, it was decided to analyze the effect of a biologically inert surface modification on the growth and colonization of an LBP in the murine GI tract. To assess this, female BALB/c mice were treated with streptomycin and introduced either biotinylated or unmodified EcN via oral gavage. At indicated timepoints, fecal pellets were collected and homogenized as a proxy for the intestinal LBP abundance, which has been previously shown is an accurate approximation in this mouse model.

The results show that colonization of the modified LBP is non-inferior to the unmodified control (FIG. 11C). For the first 72 hours, there are no significant differences between the groups and during the full 30-day window that fecal pellets were collected, the two groups demonstrated significant differences only at Day 5. Additionally, there is no significant difference between the rate of colonization, measured as the number of days for viable EcN to appear in the feces of mice, between the groups (FIG. 11D). For further evidence of the non-inferiority of surface modified LBPs, it was found that both biotinylated and unmodified LBPs stably colonized the murine GI tract at equivalent abundances out to 30-days post-gavage, as shown in FIG. 11C.

Modifications to the LBP surface are a promising method to alter their interactions with the human host and improve therapeutic efficacy. However, their use as an oral delivery strategy for LBPs remains poorly characterized. In this example, the platform of Example 1 herein was used to modify LBP surfaces and analyze critical parameters influencing their oral delivery. This work analyzed both the effect of LBP parameters (growth and biotin dilution, concentration, contact time) on the success of surface modifications as a delivery strategy, as well as the effect of the platform on measures of LBP efficacy and clinical translation (viability, toxicity, metabolism, colonization, and storage). In doing so, a pipeline has been established for the in vitro characterization of oral delivery platforms for LBPs. Using this approach, it was found that LBP growth dilutes the concentration of targeting ligand on the LBP surface, inhibiting their attachment to target proteins. In contrast, it was demonstrated that altering LBP concentration and contact time can significantly improve the attachment of modified LBPs to their target. By altering LBP concentration, it was found that the attachment of modified LBPs was inhibited at high concentrations, likely a result of steric hindrance between the targeting ligands on the LBP surface and their targets. This work further confirmed that NETS-ester-based bioconjugation does not significantly impede critical parameters known to influence LBP efficacy, including their growth, viability, metabolite secretion, and in vivo colonization. Importantly, it has been shown that surface modified LBPs can be stored for up to one week without effecting their viability or target binding, using clinically relevant storage conditions.

Collectively, the disclosure and examples herein provide a foundation of support for bioconjugation-based surface modifications and establishes both key considerations for designing oral LBP delivery systems, as well as the experimental approaches for evaluating them.

### Materials and Methods

Cell Lines and Culture. Lactobacillus casei (ATCC 393) and Bacillus coagulans (ATCC 7050) were purchased from ATCC. Escherichia coli DH5α was purchased transformed with a pBS-ldhGFP plasmid conferring GFP-expression and ampicillin resistance (selection at 100 μg/mL), a gift from Michela Lizier (Addgene plasmid #27170; http://n2t.net/addgene:27170; RRID:Addgene_27170).³⁴⁻³⁵ Escherichia coli Nissle 1917 was a gift from Nathan Crook and came transformed with a plasmid conferring kanamycin resistance (selection at 50 μg/mL).³⁶ Glycerol stocks of all bacterial strains were prepared from overnight cultures, diluted 1:1 in 50% sterile glycerol. L. casei was grown in MRS media at 37° C. under static conditions, while B. coagulans (grown in Nutrient Broth) and E. coli strains (grown in Luria Broth) were grown at 37° C. in a shaking incubator (200 rpm). All bacterial cultures were inoculated from glycerol stocks at least 12 hours before use in a study in media supplemented with appropriate concentrations of antibiotics. Caco-2 cells were purchased from the University of North Carolina at Chapel Hill Tissue Culture Facility and cultured in phenol-free Dulbecco's Modified Eagle's Medium (DMEM, Gibco) supplemented with 20% Fetal Bovine Serum (FBS) and 1% penicillin-streptomycin.

Biotinylation of Bacteria and Fluorescent Streptavidin Binding. Bacteria cultures were grown overnight and biotinylated as previously described.¹⁵ Briefly, bacteria were harvested via centrifugation, washed twice in ice-cold PBS, diluted to an optical density measured at 600 nm (OD600) of 1.0, and reacted for 20 minutes on ice at a concentration of 1 mg/mL of N-hydroxysulfosuccinimide functionalized biotin (EZ-Link Sulfo-NHS Biotin; ThermoFisher). Samples were washed 2× with PBS via centrifugation at 4,000 rpm for 10 minutes. 10 μL of fluorescent streptavidin probe (Streptavidin Alexa Fluor 594 conjugate, Invitrogen) was mixed with 100 μL of bacteria, washed as described above, and imaged using fluorescent microscopy (Revolve, Echo). Fluorescence intensity was measured using a microplate reader (Synergy H1, BioTek).

Biotin Dilution During Growth. E. coli DH5α cultures were grown overnight and biotinylated as described above. Following washes, E. coli was diluted to an OD600 of ˜0.2 and transferred to an incubator at 37° C. At each timepoint, samples were removed and used to quantify the surface biotin concentration, attachment to a streptavidin-coated plate, and bacterial concentration. A fluorescent streptavidin probe was used to calculate surface biotin concentration; the probe was incubated with samples as described above, and the number of streptavidin molecules was determined using a standard curve from the fluorescence intensity on a microplate reader (Synergy H1, BioTek). Attachment was determined by incubating samples on a streptavidin-coated plate (Pierce™ Streptavidin Coated High Binding Capacity Plate; Life Technologies) for 20 minutes while shaking at room temperature. Fluorescence intensity was measured prior to and following washes to remove unbound bacteria using a microplate reader. Images were captured on the bottom of the well plate (Echo; Revolve). Bacterial concentration was determined by plating samples on selective agar plates and enumerating colony forming units (CFU).

Attachment Studies. E. coli DH5α was grown and biotinylated as previously described. Cultures were diluted to indicated OD600 and incubated on a streptavidin-coated well plate for 20 minutes at room temperature, shaking on a microplate shaker. For the contact time study, samples were incubated at 4° C. for indicated timepoints under static conditions. For all attachment studies, wells were washed 4× with PBS to remove unbound bacteria. Fluorescence intensity was quantified on a microplate reader prior to and following washing (Synergy H1, BioTek) and three images at unique positions on the well floor were taken for each replicate (Revolve, Echo). For the growth of attached bacteria at varying concentrations, the well medium was replaced with fresh LB broth (supplemented with 100 μg/mL ampicillin) and the microplate was transferred to an incubator at 37° C. At 1-hour increments, the well plate was removed, washed 4× as described above, and images were taken of the well plate floor for quantification. Image analysis was conducted using Particle Counting in ImageJ.

Viability, Growth, and Storage. LBPs were biotinylated as previously described. For viability assessment, samples were taken immediately prior to and following biotinylation, serially diluted in PBS, plated on selective agar plates, and enumerated for viable CFUs. Samples were then diluted 1:100 in fresh medium in triplicate, added to a 96-well plate, and sealed (Breathe-Easy Sealing Membrane, Sigma). Growth curves were measured in a microplate reader (Synergy H1, BioTek) at 37° C. for 8 hours, reading absorbance at 600 nm every 10 minutes. For storage studies, LBPs were diluted 1:1 in 50% sterile glycerol in deionized water and frozen at −80° C. At indicated timepoints, vials were thawed at room temperature and CFUs were enumerated.

Mammalian Viability. Caco-2 cells were seeded in tissue culture treated 96-well plates 48 hours before use at 10,000 cells/well. L. casei and EcN cultures were grown and biotinylated as described above and diluted to an OD of 0.8 (˜10⁹ CFU/mL). 10-fold dilutions were conducted in phenol-free DMEM to achieve a range of concentrations from 10⁷-10⁹ CFU/mL, and 100 μL were added to Caco-2 wells. Cells were incubated for 1- or 2-hours, and an MTT assay was conducted according to manufacturer's instructions (Vybrant MTT Cell Proliferation Assay Kit, Invitrogen), using DMSO to solubilize formazan in the final step. The average of a triplicate of untreated controls was used as the 100% viability reference point, while the average of a triplicate of 1% Triton-X-treated cells was used as the 0% viability reference point. Viability was calculated assuming a linear relationship.

Lactic Acid Secretion. L. casei was cultured and biotinylated as previously described. Cultures were collected via centrifugation at 4,000 rpm for 5 minutes and resuspended in MRS media or M9 minimal media (5× M9 Minimal Salts, BD Difco) supplemented with 0.4% glucose, then diluted to an OD600 of 0.5. Samples were removed at t=0 and placed on ice and cultures were transferred to 37° C. in a static incubator. At indicated timepoints, samples were removed, bacteria were pelleted, and lactate concentration was assessed according to manufacturer's instructions with the supernatent (L-Lactate Assay Kit, BioAssay Systems). L. casei concentration was quantified via plating on MRS agar plates and enumerating viable CFUs.

In vivo Colonization of Modified Bacteria. Animal studies were conducted in accordance with and approved by the Institutional Animal Care and Use Committee (IACUC) of The University of North Carolina at Chapel Hill. Eight-week old female BALB/c mice were purchased from Charles River and acclimated for at least 72-hours prior to use. Mice were placed on a controlled diet (Open Standard Diet; Research Diets) for 7 days prior to the start of any studies.

Streptomycin was given ad lib in the drinking water for 24-hours (5 g/L), followed by an 18-hour wash-out period. Mice were gavaged with 100 μL of 10⁹ CFU/mL of EcN in sterile saline (10⁸ CFU total) following biotinylation, as described above, with flexible 20-gauge gavage needles (30 mm; Instech). Feces was collected from mice as previously described,¹⁵ homogenized in PBS, serially diluted and plated on kanamycin selective LB agar plates (50 μg/mL).

Statistical analysis. Statistical analyses conducted using Graphpad Prism version 8.4.3 for macOS.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A composition for enhancing gastrointestinal health comprising:
microbes modified with one or more surface moieties, the surface moieties comprising functionality for binding the modified microbes to surfaces of the gastrointestinal tract.

2. The composition of claim 1, wherein the one or more surface moieties are covalently bound to the microbes.

3. The composition of claim 1, wherein the one or more surface moieties are non-covalently bound to the microbes.

4. The composition of claim 1, wherein the surfaces of the gastrointestinal tract comprise epithelial cells, mucus, unmodified microbes, and combinations thereof.

5. The composition of claim 1, wherein the one or more surface moieties covalently bind with the surfaces of the gastrointestinal tract.

6. The composition of claim 1, wherein the one or more surface moieties non-covalently bind with surfaces of the gastrointestinal tract.

7. The composition of claim 1, wherein the surface moieties exhibit specific binding interactions with the surfaces of the gastrointestinal tract.

8. The composition of claim 7, wherein the surface moieties are selected from the group consisting of polymeric species, antibodies, peptides, aptamers, fats, metabolites, peptidomimetics, and combinations thereof.

9. The composition of claim 1, wherein the surface moieties exhibit non-specific binding interactions with the surfaces of the gastrointestinal tract.

10. The composition of claim 1, wherein the modified microbes comprise bacteria, fungi, viruses, protozoa, algae, archaea or mixtures thereof.

11. A method of treating gastrointestinal surfaces comprising:
modifying microbes with one or more surface moieties;
delivering the modified microbes to the gastrointestinal tract of an individual; and
binding the modified microbes to the gastrointestinal surfaces via the one or more surface moieties.

12. The method of claim 11, wherein the one or more surface moieties increase or enhance binding of the modified microbes to the gastrointestinal surfaces relative to one or more unmodified microbial species.

13. The method of claim 11, wherein the modified microbes block attachment of pathogenic species to the gastrointestinal surfaces.

14. The method of claim 11, wherein the one or more surface moieties are covalently bound to the microbes.

15. The method of claim 11, wherein the one or more surface moieties are non-covalently attached to the microbes.

16. The method of claim 11, wherein the surfaces of the gastrointestinal tract comprise epithelial cells, mucus, unmodified microbes, and combinations thereof.

17. The method of claim 11, wherein the one or more surface moieties covalently bind with the surfaces of the gastrointestinal tract.

18. The method of claim 11, wherein the one or more surface moieties non-covalently bind with surfaces of the gastrointestinal tract.

19. The method of claim 11, wherein the surface moieties exhibit specific binding interactions with the surfaces of the gastrointestinal tract.

20. The method of claim 19, wherein the surface moieties are selected from the group consisting of polymeric species, antibodies, peptides, aptamers, fats, metabolites, peptidomimetics, and combinations thereof.

21. The method of claim 11, wherein the surface moieties exhibit non-specific binding interactions with the surfaces of the gastrointestinal tract.

22. The method of claim 11, wherein the modified microbes comprise bacteria, fungi, viruses, protozoa, algae, archaea or mixtures thereof.

23. The method of claim 11, wherein the surface moieties are selected from the group consisting of small molecule ligands, intact cell membranes from epithelial cells and bacteria, and components of cell membranes
